(19) United States
(12) Patent Application Publication
(10) Pub. No.: US 2019/0175513 A1
(43) Pub. Date: Jun. 13, 2019

(54) ALL NATURAL, NON-TOXIC SUBLINGUAL DRUG DELIVERY SYSTEMS

(71) Applicant: SENTAR PHARMACEUTICALS, INC., Costa Mesa, CA (US)

(72) Inventor: Paul EDALAT, Irvine, CA (US)

(21) Appl. No.: 16/277,070

(22) Filed: Feb. 15, 2019

Related U.S. Application Data
(62) Division of application No. 15/613,057, filed on Jun. 2, 2017.
Application No. 15/613,057 is a continuation of application No. 14/760,311, filed on Jul. 10, 2015, now abandoned.
(60) Provisional application No. 61/937,021, filed on Feb. 7, 2014.

(51) Int. Cl. A61K 9/48; A61K 45/06; A61K 9/00

(52) U.S. Cl. CPC A61K 9/4808; A61K 9/4825; A61K 9/006; A61K 45/06

(57) ABSTRACT

Methods for producing all natural non-toxic sublingual delivery systems that have improved absorption and onset profiles for active compounds, along with improved bioavailability and pharmacokinetics compared to tablets for oral administration.

### CROSS-REFERENCE TO RELATED APPLICATIONS

This application is a divisional of U.S. application Ser. No. 15/613,057, filed on Jun. 2, 2017, which is a continuation application of U.S. patent application Ser. No. 14/760,311, filed on Jul. 10, 2015, which is a national stage entry of PCT/US2014/022054 with an International Filing Date of Mar. 7, 2014, which application claims the full Paris Convention benefit of, and priority to, U.S. provisional application serial numbered 61/937,021, filed on Feb. 7, 2014. The contents of each of these application is incorporated by this reference as if fully set forth herein in their entirety.

### BACKGROUND OF THE DISCLOSURE

It has been a longstanding need in the art to compound active agents in ways that their respective absorption and onset profiles can be enhanced. The present disclosures related to improved approaches which permit many legacy-patented products to be more effectively delivered along with newly compounded desiderata and plethoric long-term challenged moieties to finally be addressed.

Among the primary objectives of current pharmaceutical, supplement-based and nutraceuticals markets is delivery of more active ingredients by safer and more efficient means. In order to do this, research has been undertaken to employ, for example, time-release mechanisms, as well as to engineer pharmokinetic compounds to treat mammals, including humans, pets, and test subjects. However, the present inventor has taken select inventive principles to enable and reconfigure formulations to enable improved and enhanced systems to facilitate delivery of certain active ingredients whereby dosing regimens and chemical level can be attenuated to improve safety and efficacy.

Prior to the advent of the instant teachings, sublingual delivery has been constrained, and urgent and longstanding needs to compound agents to treat pulmonary hypertension, erectile dysfunction, cholesterol and blood pressure issues have not been adequately advanced.

### SUMMARY OF THE DISCLOSURES

Briefly stated, enhanced sublingual delivery systems improve absorption and onset profiles for numerous actives, along with bioavailability and pharmacokinetics results that are better than expected for families of moieties compounds and legacy-patented formulations, as desired by the marketplace.

According to embodiments, there are provided a plurality of compressed dry powder sublingual delivery vehicles effective for delivery of active agents to mammals. These include pharmaceuticals, nutraceuticals, supplements, and pet products, inter alia.

According to embodiments, there is provided a novel enhanced continuous sublingual capsule extrusion process, which comprises, in combination; extruding at least an eccentric gelatin capsule case; extruding at least a gelatin plug set; filling the extrudates; and, plugging the same respective with gelatin plugs; whereby the process is continuous, capsule diameter sets with extrusion die; length of capsules is determined by final cutting steps; and, the eccentric nature of resultory capsules provides for a thin wall to enable at least one of dissolution, or with additional processing, other mechanisms of action.

According to embodiments, there is provided a novel enhanced continuous sublingual capsule extrusion process, delivering at least one of vasodilators, cholesterol management tools, and agents for treating blood pressure, inter alia.

According to embodiments, there is provided a novel enhanced continuous sublingual capsule extrusion process, effective for delivering lower levels of active ingredients than conventionally thought to be effective.

According to embodiments, there is provided there is provided a continuous offset extruded gel strip process, comprising, in combination: extruding at least an offset gelatin strip; extruding at least pairing sets of gelatin plugs; extending the filling; and, finishing the gelatin cap extrusions.

According to embodiments, there is provided a process which is continuous, has strip dimensions set with extrusion dies; wherein the strip is cut to a desired length at end of certain processes; and, the offset strip causes dissolution of the thin wall to enable improved delivery of active ingredients to mammals.

### BRIEF DESCRIPTION OF THE DRAWINGS

FIGS. 1A through 1D are select alternate compressed dry powder sublingual tablet shapes according to aspects of the present invention.

FIGS. 2A through 2C illustrate select embodiments relating to continuous sublingual capsule extension.

FIGS. 3A through 3C schematically illustrate offset gel strip extension processor according to the instant teachings.

FIGS. 4A and 4B schematically illustrate alternative finishing processes.

FIGS. 5A and 5B depict schematics and process steps for sublingual waffle gel strips and dimpled gel strips according to embodiments of the present inventions.

FIGS. 6A, 6B, and 6C further illustrate embodiments according to the present inventions.

FIGS. 7A and 7B further illustrate embodiments and processes to create the waffle gel strips, according to the present inventions.

FIGS. 8A and 8B likewise schematically illustrate novel enhanced processes and methodologies according to the present inventions.

FIG. 9 is a table depicting an exemplary embodiment according to the teachings of the present inventions.

### DETAILED DESCRIPTION

The present inventor has formulated and tested numerous approaches to improvements in absorption and onset functions of plethoric groups and families of compounds. The Appendix to the provisional application which is a priority basis of the instant filing comprises a list of formulations licensed by the FDA and California Dept. of Health to be manufactured, wholesaled, and/or repackaged by the present inventor/assignee. Many of these chemical entities, compounds and families have been profiled, and research has demonstrated unexpected benefits of delivering them sublingually. Accordingly, the present inventor has tested and formulated lower dosages of select compounds and achieved unexpectedly better results—as explained herein, and claimed below.

Among those moieties best served by sublingual approaches to bioavailability improvements are exemplary compounds and other common agents used for pulmonary hypertension, blood pressure, cholesterol issues, and vasodilation. Without limiting the observed improvements to one mechanism of action, the present inventor has extended research into related areas ranging from the above listed to another phosphodiesterase-5 (PDE-5) inhibitors to medications for diabetes.

Expressly incorporated by this reference, as if fully set forth herein in their entirety, are the following patents and publications: U.S. Pat. Nos. 5,260,440; 6,316,460; 6,002,021; 4,444,784; 5,159,104; 6,100,407; EP 1 171 134; PCT/US2000/00662; U.S. Pat. Nos. 8,497,370; 7,279,457, 3,428,728; 8,201,503; EP 1 019 039; US 2014/0011755; US 2013/0143894; US 2013/0059854; US 2010/0209359; US 2010/0113453; US 2010/0069397; US 2007/0122355; US 2006/0099300; US 2003/0073133; US 2003/0022912; U.S. Pat. Nos. 8,293,295; 7,449,175; 7,329,416; 7,258,850; 6,903,127; 6,632,419; 6,592,850; 6,552,024; 6,548,490; 6,531,114; 6,428,769; 6,403,597; 6,342,251; 6,211,156; 6,200,591; WO 2005/039530A1; WO 00/54777A1; EP 2,452,675A1; EP 1,536,769A2; EP 960,921A2; EP 1,171,134A1; DE 19834505A1; AU 3744800A; CN101683325A; CN10157930A; CN100488509C; CN101224222A; CN101057850A; U.S. Pat. Nos. 8,563,534; 8,501,715; 8,481,570; 8,211,922; 8,158,611; 7,279,459; 7,186,704; GB 2497728A; CN 101991854A; U.S. Pat. Nos. 8,012,503; 7,163,705; CN 001600159A; US 2013/0123354; U.S. Pat. No. 7,138,107; and, U.S. Pat. No. 6,849,649.

The previously available controlled release sublingual tablet formulations had a number of deficiencies. The present invention addresses these deficiencies. This invention as described is particularly applicable to a number of compounds, as shown by work done with, for example, extremely low dosages of active ingredients such as sildenafil. The practice of this invention using sub-compounds is desired since increasing the bioavailability of this drug is useful in the treatment of pulmonary hypertension, and psychogenic impotence. Further, this invention allows for the successful use of lower concentrations of this drug without major side effects occurring which are extremely undesirable.

It is known from in vitro studies that sildenafil is approximately 4,000 fold more selective for inhibiting phosphodiesterase type 5 (PDE5) than on other known phosphodiesterases, such as PDE3, which is involved in control of cardiac contractility. Sildenafil is reportedly only about 10-fold as potent for PDE5 compared to PDE6, an enzyme found in the retina and it is this lower selectivity which is thought to be the basis for abnormalities related to color vision observed with higher doses or plasma levels.

Generally, sublingual dosage forms dissolve within a time period of at least about 2 minutes, but less than about 7 minutes. Dissolution time in water for the presently contemplated dosage forms ranges from about 3 minutes to about 5 minutes.

Formulations including an active agent, such as insulin, and one or more excipients, such as a chelator and/or solubilizing agent, that dissolve rapidly in aqueous media are likewise described herein, and contemplated by the instant teachings. In select embodiments, the formulations are suitable for subcutaneous or sublingual administration. These formulations are rapidly absorbed through mucosal surfaces (parenteral, pulmonary, etc.) and through the fatty tissue when administered subcutaneously. This is achieved through the addition of excipients, especially solubilizers such as acids and metal chelators.

As generally used herein, a drug is considered “highly soluble” when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. A drug is considered highly soluble when 90% or more of an administered dose, based on a mass determination or in comparison to an intravenous reference dose, is dissolved. Solubility can be measured by the shake-flask or titration method or analysis by a validated stability-indicating assay.

As generally used herein, an immediate release drug formulation is considered “rapidly dissolving” when no less than 85% of the labeled amount of the drug substance dissolves within 30 minutes, using U.S. Pharmacopeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

Although described with reference to small-molecule drugs like insulin, the instant formulations may be used with other agents, including peptides, proteins, nucleotide molecules (RNA sequences, DNA sequences), sugars, polysaccharides, and small organic molecules. In some examples, the active agent is at least slightly soluble in aqueous medium (i.e. 10,000 parts of aqueous solvent per solute), and in others, is highly soluble in aqueous medium. Preferably the active agent is highly potent, so that only a small amount (e.g. in the microgram range) is needed to provide a therapeutic effect. Suitable peptides include but are not limited to insulin and derivatives of insulin, such as lispro; C-peptide; glucagon-like peptide 1 (GLP 1) and all active fragments thereof; human amyl in and synthetic forms of amyl in, such as pramlintide; parathyroid hormone (PTH) and active fragments thereof (e.g. PTH1-34); calcitonin; human growth hormone (HGH); erythropoietin (EPO); macrophage-colony stimulating factor (M-CSF); granulocyte-macrophage-colony stimulating factor (GM-CSF); and interleukins. In the preferred embodiment the active agent is insulin. Suitable small molecules include nitroglycerin, sumatriptan, narcotics (e.g. fenatnyl, codeine, propoxyphene, hydrocodone, and oxycodone), benzodiazepines (e.g. Alprazolam, Clobazam, Clonazepam, Diazepam Flunitrazepam, Lorazepam, Nitrazepam, Oxazepam, Temazepam, and Triazolam), phenothiazines (Chlorpromazine, Fluphenazine, Mesoridazine, Methotrimeprazine, Pericyazine, Perphenazine, Prochlorperazine, Thioproperazine, Thioridazine, and Trifluoperazine), and selective serotonin reuptake inhibitors (SSR1s) (e.g. sertraline, fluvoxamine, fluoxetine, citalopram, and paroxetine).

The dosages of the active agents depend on their bioavailability and the condition, ailment, disease or disorder to be treated. The compositions optionally contain one or more excipients.

In select embodiments, one or more solubilizing agents are included with the active agent to promote rapid dissolution in aqueous media. Suitable solubilizing agents include wetting agents such as polysorbates and poloxamers, non-ionic and ionic surfactants, food acids and bases (e.g. sodium bicarbonate), and alcohols, and buffer salts for pH control. Suitable acids include acetic acid, ascorbic acid, citric acid, and hydrochloric acid. For example, if the active agent is insulin, a preferred solubilizing agent is citric acid, as known to those skilled in the art.

Diluents, also referred to herein as fillers, are typically necessary to increase the bulk of a solid dosage form so that a practical size is provided for compression of tablets or formation of beads and granules. Suitable fillers include, but are not limited to, dicalcium phosphate dihydrate, calcium sulfate, lactose, sucrose, mannitol, sorbitol, cellulose, microcrystalline cellulose, powdered cellulose, kaolin, sodium chloride, dry starch, hydrolyzed starches, pregelatinized starch, silicone dioxide, titanium oxide, magnesium aluminum silicate, calcium carbonate, compressible sugar, sugar spheres, powdered (confectioner's) sugar, dextrates, dextrin, dextrose, dibasic calcium phosphate dehydrate, glyceryl palmitostearate, magnesium carbonate, magnesium oxide, maltodextrin, polymethacrylates, potassium chloride, talc, and tribasic calcium phosphate.

Binders are used to impart cohesive qualities to a solid dosage formulations, and thus ensure that a tablet, bead or granule remains intact after the formation of the dosage forms. Suitable binder materials include, but are not limited to, starch, pregelatinized starch, gelatin, sugars (including sucrose, glucose, dextrose, lactose and sorbitol), dextrin, maltodextrin, zein, polyethylene glycol, waxes, natural and synthetic gums such as acacia, guar gum, tragacanth, alginate, sodium alginate, celluloses, including hydroxypropylmethylcellulose, carboxymethylcellulose sodium, hydroxypropylcellulose, hydroxylethylcellulose, ethylcellulose, methyl cellulose, and veegum, hydrogenated vegetable oil, Type I, magnesium aluminum silicate, and synthetic polymers such as acrylic acid and methacrylic acid copolymers, carbomer, methacrylic acid copolymers, methyl methacrylate copolymers, aminoalkyl methacrylate copolymers, polyacrylic acid/polymethacrylic acid, and polyvinylpyrrolidone.

Lubricants are used to facilitate tablet manufacture. Examples of suitable lubricants include, but are not limited to, magnesium stearate, calcium stearate, stearic acid, glyceryl behenate, glyceryl monostearate, glyceryl palmitostearate, hydrogenated castor oil, hydrogenated vegetable oil, type I, sodium benzoate, sodium lauryl sulfate, sodium stearyl fumarate, polyethylene glycol, talc, zinc stearate, and mineral oil and light mineral oil.

Stabilizers are used to inhibit or retard drug decomposition reactions which include, by way of example, oxidative reactions. A number of stabilizers may be used.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate.

If desired, the tablets, wafers, films, lozenges, beads, granules, or particles may also contain minor amount of nontoxic auxiliary substances such as dyes, masking agents, sweeteners, coloring and flavoring agents, pH buffering agents, or preservatives.

Blending or copolymerization sufficient to provide a certain amount of hydrophilic character can be useful to improve wettability of the materials. The active compounds (or pharmaceutically acceptable salts thereof) may be administered in the form of a pharmaceutical composition wherein the active compound(s) is in admixture or mixture with one or more pharmaceutically acceptable carriers, excipients or diluents. Suitable dosage forms include powders, films, wafers, lozenges, capsules, and tablets. Following administration, the dosage form dissolves quickly releasing the drug or forming small particles containing drug, optionally containing one or more excipients.

Select variations of the instant formulations may dissolve in a time period ranging from 1 second to at least about 3 minutes, 3 to 5 minutes, 5 to 8 minutes, or 8 to 12 minutes. One formulation's dissolution time is less than 30 seconds. According to the instant teachings, the drugs are absorbed and transported to the plasma quickly, resulting in a rapid onset of action (for example, beginning within about 5 minutes following administration and peaking at about 15-30 minutes following administration).

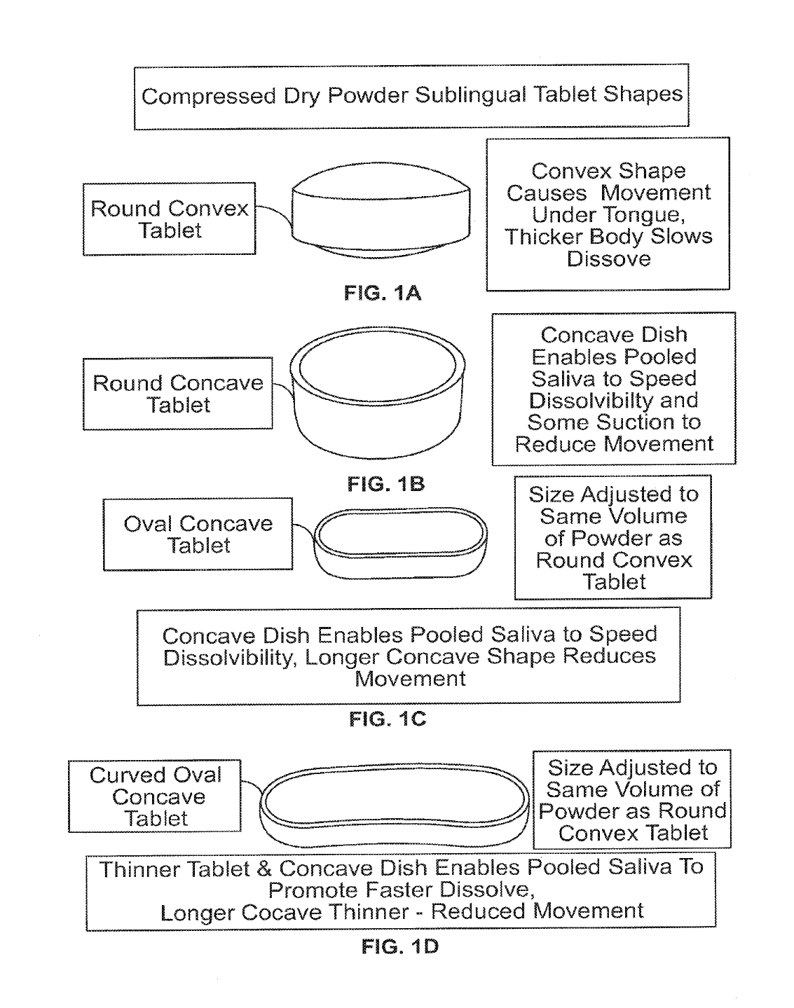

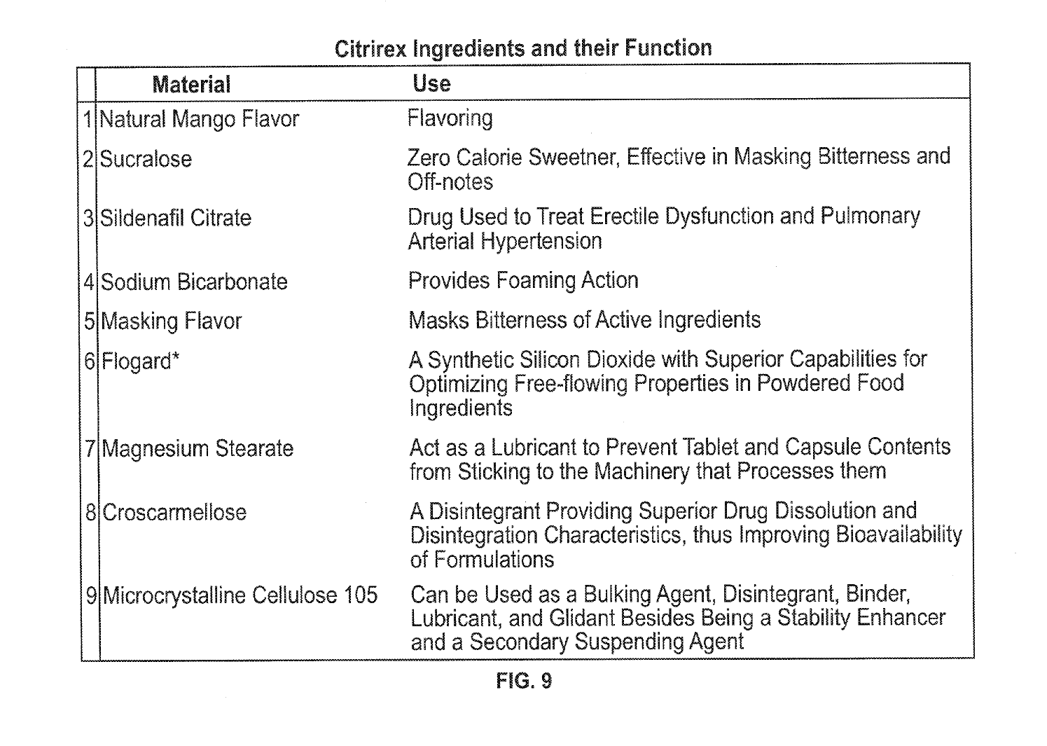

FIG. 9 shows an improved formulation for the CITRIREX™ brand of compound (select formulations for export only, Scilabs Pharmaceuticals, Irvine, Calif. 92614, FDA drug labeler code 54317). The present inventor has been able to step down dosage requirements along with overcoming bitterness/gustatory issues, using processes illustrated in FIGS. 1-8B.

By way of further example of the benefits of the instant teachings as applied to treating pulmonary hypertension, extremely low dosages of compounds like sildenafil can be efficacious, have lower risk profiles, and may have other and further advantages when delivered with all natural vehicles and systems.

It is known that oral medicines are particularly desirable and sought after discreet form of treatment for sexual dysfunction. Recently, the oral use of the citrate salt of sildenafil has been approved by the U.S. Food and Drug Administration (FDA) for the treatment of male erectile dysfunction. Sildenafil is reported to be a selective inhibitor of cyclic-GMP-specific phosphodiesterase type 5 (PDE5), the predominant isozyme metabolizing cyclic GMP formed in the corpus cavernosum. Since sildenafil is a potent inhibitor of PDE5 in the corpus cavernosum, it is believed to enhance the effect of nitric oxide release. Inasmuch as sildenafil at the currently recommended doses of 25-100 mg has little effect in the absence of sexual stimulation, sildenafil is believed to restore the natural erectile response to sexual stimulation but not cause erections in the absence of such stimulation. The localized mechanism by which cyclic GMP stimulates relaxation of the smooth muscles has not been elucidated.

In dose-response studies, increasing doses of sildenafil (25 to 100 mg) reportedly increased the erectogenic efficacy of sildenafil. However, the oral administration of sildenafil is also accompanied by dose-responsive undesirable side effects, including more serious side effects, such as syncope (loss of consciousness), priapism (erection lasting 4 hours or more) and increased cardiac risk (coital coronaries). It is noted these can be brought on in some cases by physiological predisposition, adverse drug interaction or potentiation, or by drug abuse. In particular, hypotension crisis can result from the combination of sildenafil citrate and organic nitrates, causing, in some cases death, so its administration to patients who are concurrently using organic nitrates (such as nitroglycerin) in any form is contraindicated. Thus, there is a need and desire for oral administration forms that promote the bioavailability of sildenafil at lower doses while minimizing side effects.

Early stage sublingual tablets are well documented in the literature since the beginning of this century. The main reason for sublingual route of drug administration is to provide a rapid onset of action of potent drugs. Another reason is to avoid the first pass metabolism by the liver.

The term “controlled release” when applied to sublingual tablets is limited to a maximum of about 60 minutes. Traditional sublingual tablets are usually designed as water soluble tablets made of water soluble sugars such as sorbitol, lactose, mannitol, etc. In the literature, controlled release sublingual tablets are very scarce. U.S. Pat. No. 3,428,728 to Lowey (1969) describes a controlled release sublingual tablet made by cooking gum acacia and sorbitol (by heating) till partial dryness followed by addition of citric acid, color and flavor followed by cooling. Active ingredients such as nitroglycerin, caffeine, guaiocolate, amylase or isoproterenol were then added to the pourable paste that was cast into tablets. However, Lowey's discovery cannot be applied to make tablets by compression. The time of release for a pharmaceutical preparation is critical to the effectiveness of the drug. The sublingual tablet of the present invention can be prepared by compression methods and provides a controlled drug release, in contradistinction to the prior art.

Therefore, the Sildenafil-analogues including Sildenafil, Homosildenafil, Hydroxyhomosildenafil, Desmethylsildenafil, Acetidenafil, Vardenafil and Udenafil, are interesting given the delivery system of the instant teachings. The Sildenafil may represent those seven compounds, may react with Statin derivative, y-polyglutamic acid derivative, Vitamin or sodium CMC to form the monoquaternary amine complex salts of Sildenafil-analogues and amine complex salts of Udenafil-analogues. Thereby, Sildenafil-analogues may represent Sildenafil, Homosildenafil, Hydroxyhomosildenafil, Desmethylsildenafil, Acetidenafil, Vardenafil and Udenafil. The involved piperazine or amine moiety, and the statins, y-polyglutamic acid derivative, Vitamin or sodium CMC may represent ostensive or potential combinations effective for sublingual delivery in accordance with the instant teachings.

Thus, the lactone ring, ester and protected derivatives of the Statins are available to prepare the above Sildenafil-analogues monoquaternary amine complex salts or Udenafil-analogues amine complex salts deliverable according to the instant teachings.

Likewise, Statins derivative and y-polyglutamic acid derivative, Vitamin or sodium CMC separately react with the piperazine group of Sildenafil-analogues or pyrollidinyl group of sildenafil-analogues to prepare the Sildenafil-analogues monoquaternary complex salts or sildenafil-analogues amine complex salts. Preferred Statins derivative are selected from Atorvastatin, Lovastatin, Pitavastatin, Rosuvastatin and Simvastatin, y-polyglutamic acid derivative are selected from alginate sodium, the y-polyglutamic acid, the sodium polyglutamate, and the GLT is referred as the copolymer of Lysine, Glutamate and Tyrosine, and the calcium polyglutamate-alginate sodium, Vitamin is selected from Retinoic Acid, Ascorbic acid, Folic acid, Gamma-Linolenic Acid, nicotinic Acid and Pantothenic acid. Thereby, the Sildenafils-y-Polyglutamic Acid, Sildenafils-Simvastatinic Acid, Sildenafils-Pramastatinic Acid, Sildenafils-Lovastatinic Acid, Sildenafils-Pitavastatin, Sildenafils-Rosuvastatin SildenafilL-Arginine, Sildenafil-CMC, Sildenafil-Mevastatinic acid, Sildenafil-Rosuvastatinic acid, Sildenafils-Lovastatinic Acid, Udenafil-CMC, Udenafil-nicotinic Acid and Udenafil-L-Retinoic Acid are obtained.

The term excipients or “pharmaceutically acceptable carrier or excipients” and “bio-available carriers or excipients” above-mentioned include any appropriate compounds known to be used for preparing the dosage form, such as the solvent, the dispersing agent, the coating, an anti-bacterial or anti-fungal agent and a preserving agent or the delayed absorbent. Usually, such kind of carrier or excipient does not have therapeutic activity itself. Each formulation prepared by combining the derivatives disclosed in the present invention and the pharmaceutically acceptable carriers or excipients will not cause the undesired effect, allergy or other inappropriate effects while being administered to an animal or human. Accordingly, the derivatives disclosed in the present invention in combination with the pharmaceutically acceptable carrier or excipients are adaptable in the clinical usage and in the human. A therapeutic effect can be achieved by using the dosage form in the present invention by sublingual administration. About 0.1 mg to 10 mg per day of the active ingredient is administered for the patients of various diseases.

Currently commercially available Statins widely include Atorvastatin, Cerivastatin, Fluvastatin, Lovastatin, Mevastatin, Pravastatin, Rosuvastatin and Simvastatin. The chemical names of various Statins which may be included within the scope of the instant teachings comprise: Lovastatin (disclosed in U.S. Pat. No. 4,231,938) and Simvastatin (disclosed in U.S. Pat. No. 4,444,784) can be used. Pravastatin (disclosed in U.S. Pat. No. 4,346,227) is administered as the sodium salt. Fluvastatin (disclosed in U.S. Pat. No. 4,739,073) and Cerivastatin (disclosed in U.S. Pat. Nos. 5,006,530 and 5,177,080), also administered as the sodium salts, are entirely synthetic compounds that are structurally distinct from a kind of drug to which the fungal derivatives including a hexahydronaphthalene ring belong.

The structure of commercial Statin calcium salt includes two molecules of Statins and one molecule of calcium. The so-called hemicalcium salt is referred to a combination of one molecule of Statins and one molecule of calcium. Rosuvastatin, its calcium salt and its lactone form are disclosed in U.S. Pat. No. 5,260,440, which obtains the methyl ester of Rosuvastatin under reflux followed by reduction with NaBH₄. Further, the ester is then hydrolyzed with sodium hydroxide in ethanol solution at room temperature, followed by removal of the ethanol and addition of ether, to obtain the sodium salt of Rosuvastatin. Additionally, the Rosuvastatin composition disclosed in U.S. Pat. No. 6,316,460 includes a multivalent phosphate salt of Rosuvastatin. According to the process of the present invention, dissolved Rosuvastatin sodium salt in water under a nitrogen atmosphere, and added into the Sildenafils, followed by the precipitation and crystallization, the Sildenafils-Rosuvastatinic acid monoquarternary piperazium complex salt is formed, according to embodiments.

Statins can be prepared through an intermediate in which one or both of the hydroxyls in the diol pentanoic acid group (open-ring form) or the hydroxyl of the lactone group (ring-close form) is protected via a hydrolyzable protecting group and the carboxyl group is protected via an ester derivative. U.S. Pat. No. 5,260,440 discloses the preparation of Rosuvastatin. U.S. Pat. Nos. 6,002,021 and 4,444,784 disclose a process for preparing Simvastatin, which uses the cyclic protecting group such as the dioxane, the cyclic sulfate, the cyclic phosphate and the borylidene to substitute the alkyl or aryl timely. Additionally, WO 95/13283 discloses the boric acid as the protecting group, the U.S. Pat. No. 5,159,104 discloses an esterification proceeded by the acetic anhybride and U.S. Pat. No. 6,100,407 also discloses some protecting groups.

As discussed, possible agents to be combined include Statins selected from the group consisting of Atorvastatin, Lovastatin, Pitavastatin, Rosuvastatin and Simvastatin, and the Statin structure of those drugs are hydrolized by metallic hydroxide, such as sodium, potassium, calcium, and ammonia hydroxide, and acids useful to hydrolyze the ester group of Statin.

The formation of Sildenafils-Statinic acid complex from Sildenafils HCl salt is easily obtained by reacting Sildenafils HCl with the equal molar sodium hydroxide in the presence of hydrolyzable Statins or Statins ester and derivatives. The sodium ion precedes the equal molar neutralization can take place within the HCl part of Sildenafils HCl, and the resulted NaCl is dissolved in the hydrated alcohol solution. The Statin shows the ionic state, the free state or being mixed with other unreacted ester derivative of the statin in a mixing solution of water and C1-C4 lower alcohol (i.e., the ethanol and the isopropanol). By following the amount of each Statin derivative hydrolyzed by the sufficient amount of sodium hydroxide, the term “sufficient amount of piperazium group or pyrollidinyl group” is about the amount of equal mole.

Referring now to FIGS. 1A, 1B, 1C, and 1D, compressed powder sublingual shaping morphology is offered for consideration. Both round convex and round concave tablets are shape-advantaged forms which, respectively, cause movement under the tongue, thicker body slows dissolution (convex) and enables pooled saliva to speed dissolvability along with a modicum of suction to reduce movement (concave).

Referring more specifically to FIGS. 1C and 1D, the more elliptical oral concave tablet provides dish-like structure which pools saliva, speeding dissolvability. The elongated shape likewise reduces movement. The curved oval concave tablet is size adjusted to same volume of powder as round convex tablets, yet (FIG. 1D) has further morphological advantage with respect to fictional engagement of user to reduce movement owing to elongated shape, while have the same pooled saliva advantages discussed above.

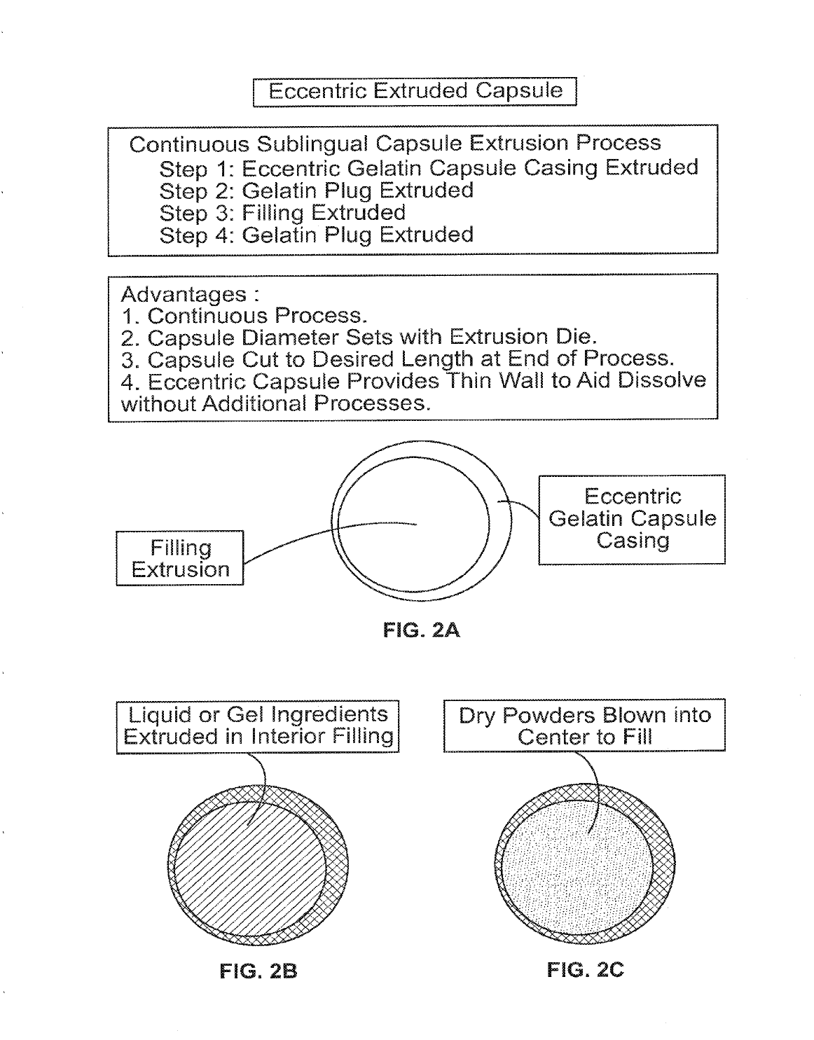

Referring now to FIGS. 2A, 2B, and 2C, eccentric extruded capsules are taught, made by a continuous process as depicted in the figures, namely;

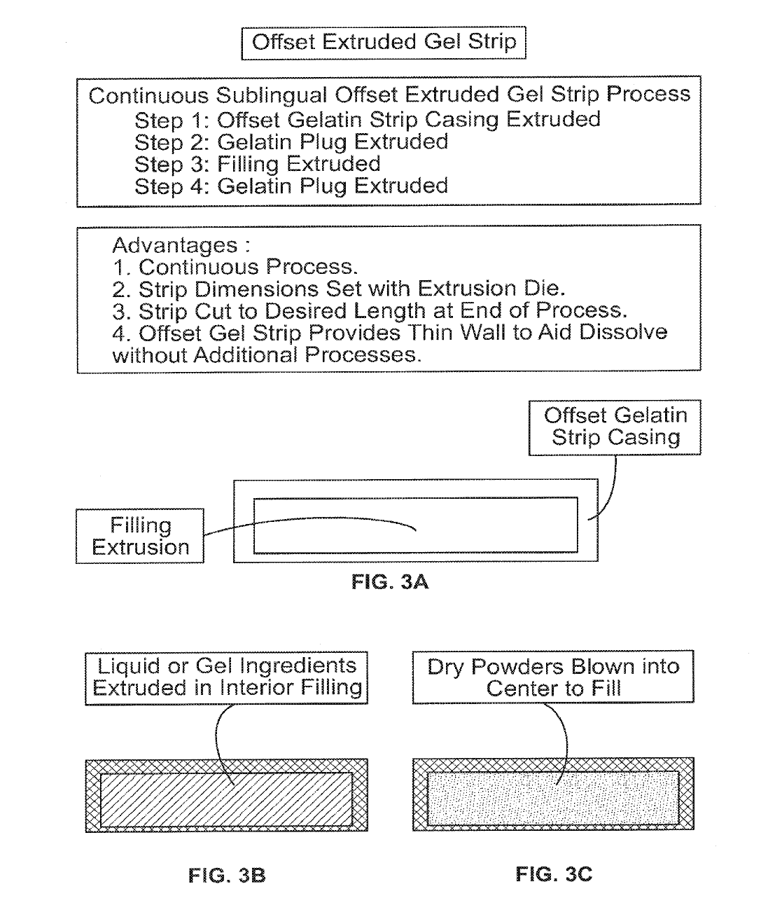

Likewise, FIGS. 3A, 3B and 3C show the process for offset extruded gel strips, as discussed above and claimed below.

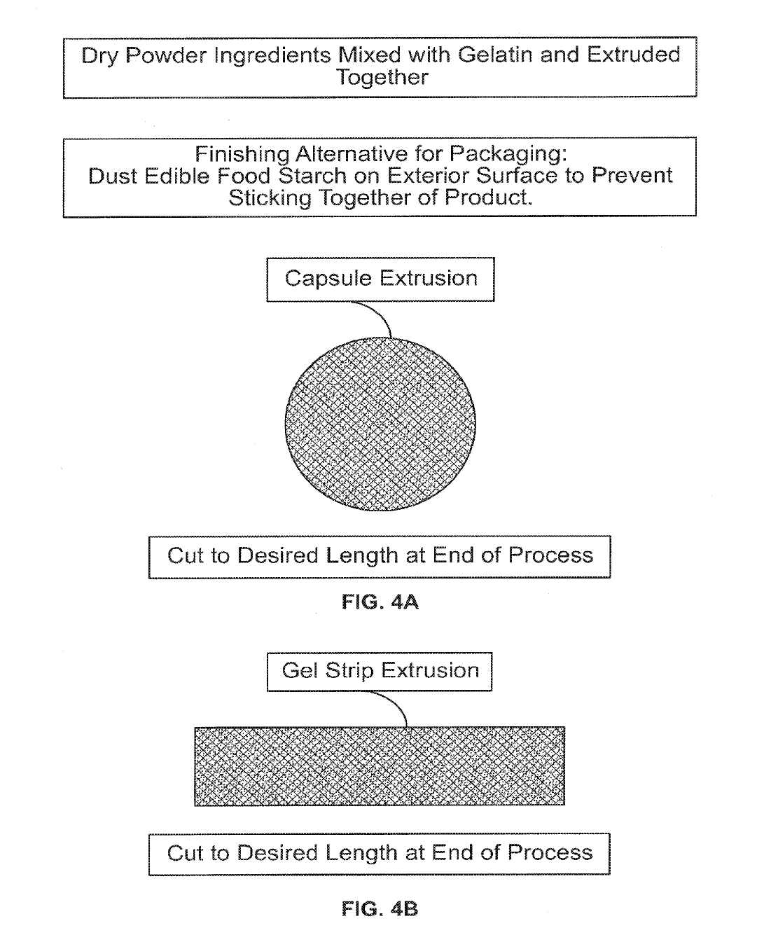

FIGS. 4A and 4B demonstrate another finishing alternative for packing, wherein dry powder ingredients are mixed together with gelatin and extruded together.

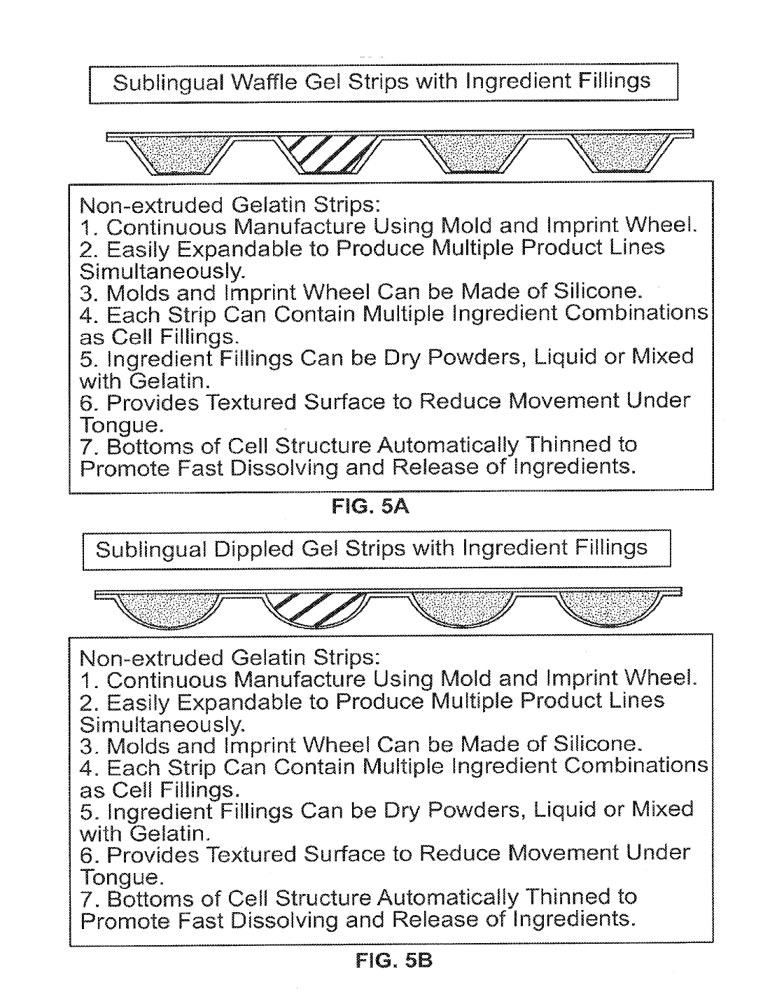

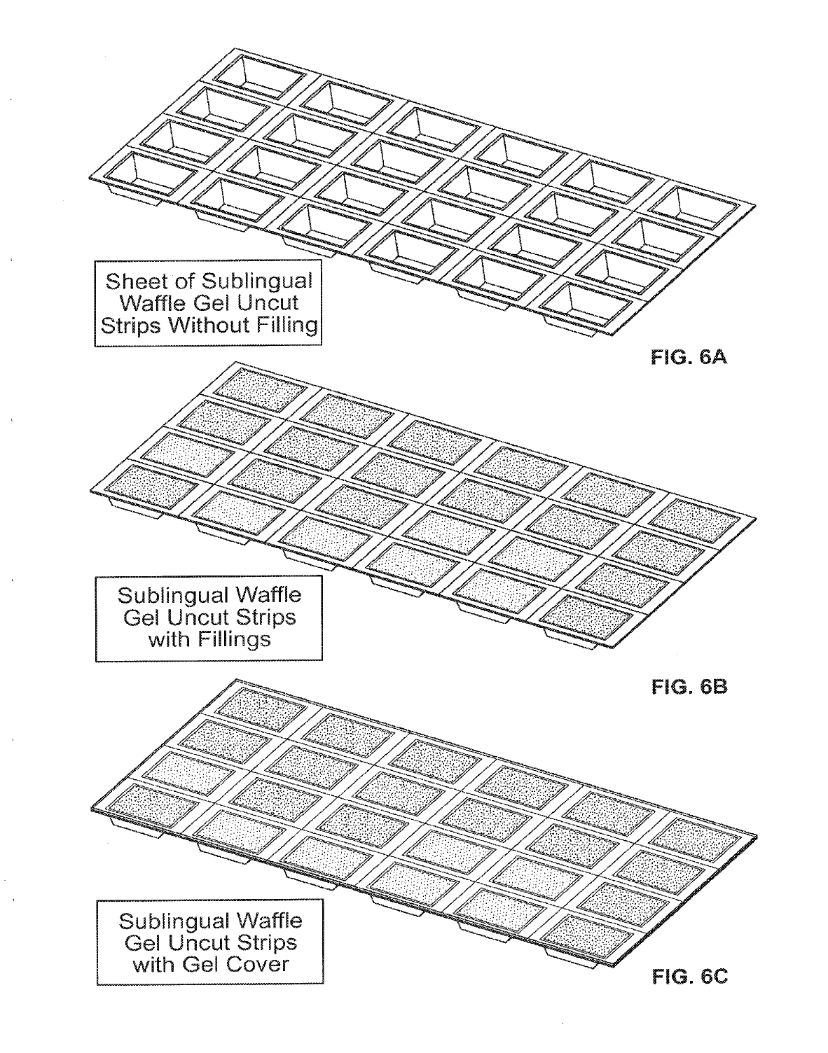

FIGS. 5A, 5B and 6A through 6C likewise illustrate processes for making sublingual waffle gel strips with active ingredient fillings.

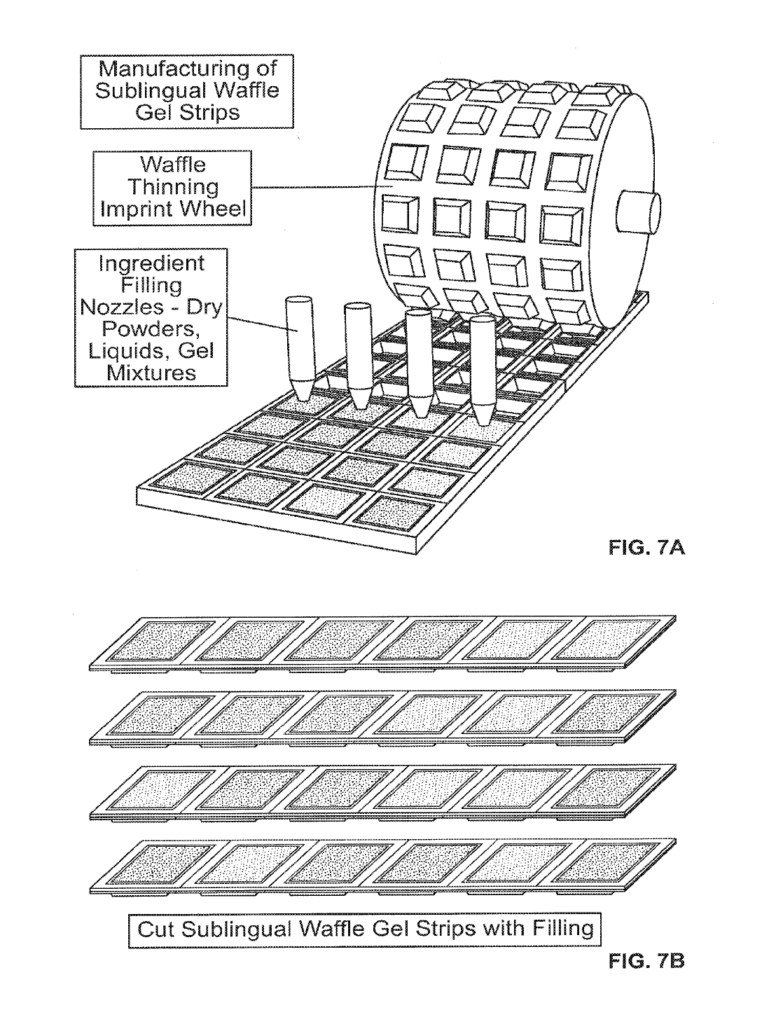

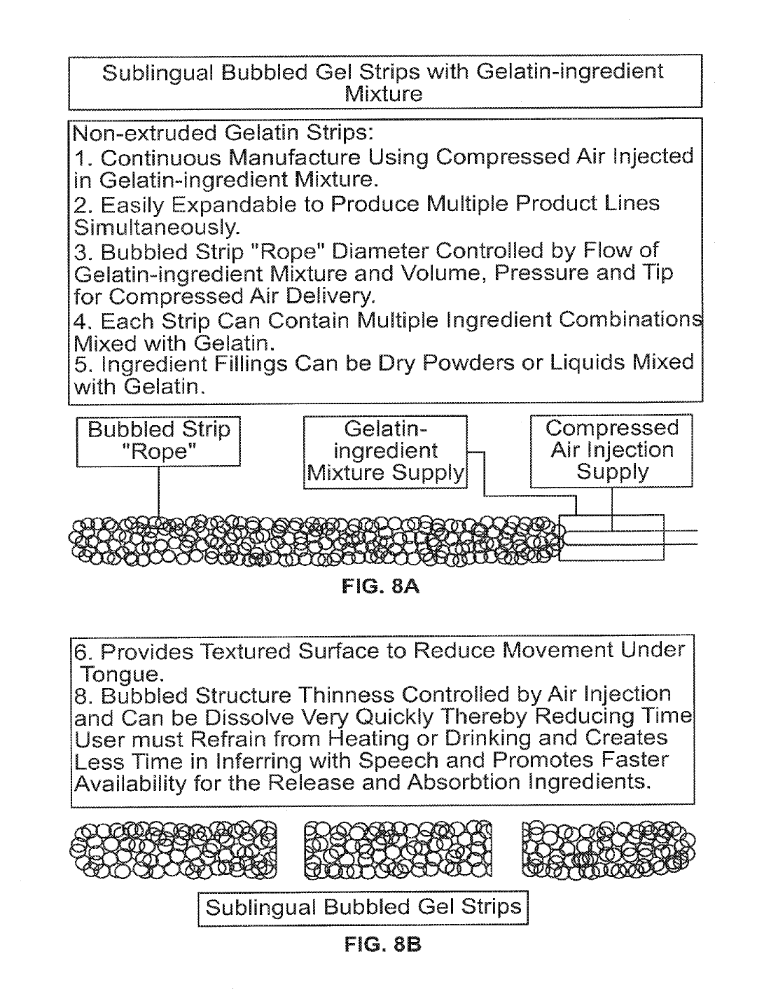

FIGS. 7 through 8, and all subparts likewise depict sublingual processes according to the present inventions, as known to those skilled in the art.

Numerous compounds formulated according to the instant process have been formulated for those in need and others can be made so based upon the processes perfected herein.

While the method and apparatus have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention. It should be understood that this disclosure is intended to yield a patent covering numerous aspects of the invention both independently and as an overall system and in both method and apparatus modes.

Further, each of the various elements of the invention and claims may also be achieved in a variety of manners. This disclosure should be understood to encompass each such variation, be it a variation of an embodiment of any apparatus embodiment, a method or process embodiment, or even merely a variation of any element of these.

Particularly, it should be understood that as the disclosure relates to elements of the invention, the words for each element may be expressed by equivalent apparatus terms or method terms—even if only the function or result is the same.

Such equivalent, broader, or even more generic terms should be considered to be encompassed in the description of each element or action. Such terms can be substituted where desired to make explicit the implicitly broad coverage to which this invention is entitled.

It should be understood that all actions may be expressed as a means for taking that action or as an element which causes that action.

Similarly, each physical element disclosed should be understood to encompass a disclosure of the action which that physical element facilitates.

Any patents, publications, or other references mentioned in this application for patent are hereby incorporated by reference. In addition, as to each term used it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans and the Random House Webster's Unabridged Dictionary, latest edition are hereby incorporated by reference.

Finally, all references listed in the Information Disclosure Statement or other information statement filed with the application are hereby appended and hereby incorporated by reference; however, as to each of the above, to the extent that such information or statements incorporated by reference might be considered inconsistent with the patenting of this/these invention(s), such statements are expressly not to be considered as made by the applicant.

In this regard it should be understood that for practical reasons and so as to avoid adding potentially hundreds of claims, the applicant has presented claims with initial dependencies only.

Support should be understood to exist to the degree required under new matter laws—including but not limited to United States Patent Law 35 USC 132 or other such laws—to permit the addition of any of the various dependencies or other elements presented under one independent claim or concept as dependencies or elements under any other independent claim or concept.

To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, the use of the transitional phrase “comprising” is used to maintain the “open-end” claims herein, according to traditional claim interpretation. Thus, unless the context requires otherwise, it should be understood that the term “comprise” or variations such as “comprises” or “comprising,” are intended to imply the inclusion of a stated element or step or group of elements or steps but not the exclusion of any other element or step or group of elements or steps.

Such terms should be interpreted in their most expansive forms so as to afford the applicant the broadest coverage legally permissible.

1. A method for producing a quick-dissolve sublingual tablet, comprising:
formulating a mixture comprising a flavoring agent, sodium bicarbonate, citric acid, and a pharmaceutically active component in powder form; and
compressing the mixture to form sublingual tablets that dissolve in less than 30 seconds upon sublingual administration.

2. The method of claim 1, wherein the sublingual tablets are round convex or round concave tablets.

3. The method of claim 1, wherein the sublingual tablet has a concavity that enables pooled saliva to speed dissolution upon sublingual administration.

4. The method of claim 1, wherein the sublingual tablets further comprise magnesium stearate, and microcrystalline cellulose.

5. The method of claim 1, wherein the sublingual tablets further comprise croscarmellose.

6. The method of claim 1, wherein the sublingual tablets further comprise microcrystalline cellulose.

7. The method of claim 1, wherein the sublingual tablets further comprise sucralose.

8. The method of claim 1, wherein the pharmaceutically active component comprises sildenafil or a sildenafil-analogue.

9. The method of claim 1, wherein the pharmaceutically active component comprises sildenafil citrate.

10. The method of claim 1, wherein the pharmaceutically active component is a statin.

11. The method of claim 1, wherein the sublingual tablets further comprise a vegetable oil.

12. The method of claim 1, wherein the sublingual tablets are formulated for rapid onset of action beginning within 5 minutes of sublingual administration.

13. The method of claim 1, wherein the compression methods provide for controlled release of the pharmaceutically active component.

14. The method of claim 1, wherein the sublingual tablets are formulated for rapid onset of action beginning within 5 minutes of sublingual administration, and peaking between 15 and 30 minutes after administration.
